(19) United States
(12) Patent Application Publication
(10) Pub. No.: US 2023/0000370 A1
(43) Pub. Date: Jan. 5, 2023

(54) PHOTOPLETHYSMOGRAPHIC BLOOD PRESSURE MEASURING DEVICE WITH REMOVABLE FINGER CUFF

(71) Applicant: PULSION MEDICAL SYSTEMS SE, Feldkirchen (DE)

(72) Inventors: Aaron Weber, Markt Schwaben (DE); André Hein, Esslingen am Neckar (DE); Thomas Thalmeier, Dorfen (DE)

(73) Assignee: PULSION MEDICAL SYSTEMS SE, Feldkirchen (DE)

(21) Appl. No.: 17/780,954

(22) PCT Filed: Nov. 30, 2020
(86) PCT No.: PCT/EP2020/083963

§ 371 Date: May 27, 2022

(30) Foreign Application Priority Data
Dec. 1, 2019 (DE) 20 2019 004 899.5

(51) Int. Cl. A61B 5/021; A61B 5/022; A61B 5/0225; A61B 5/00

(52) U.S. Cl. CPC A61B 5/02141; A61B 5/02241; A61B 5/02255; A61B 5/6826; A61B 5/70; A61B 2560/0425

(57) ABSTRACT

The blood pressure measuring device (1) is designed as a photoplethysmographic measuring system which functions according to the “vascular unloading technique”. Metrological components and a pressure generating and pressure control system are accommodated in the base part (3). The fitted cuff part (2) has an ergonomic palm support (4), two finger supports (6) divided from one another by a web (5), and also a receiving part (7) which comprises two receiving tubes (9) for fingers. The cuff part (2) projects beyond the base part (3), both towards the front and also towards the rear, and covers the base part (3) almost completely, i.e. by more than 90%. The lateral attachment of the cable (8) facing in the direction of the forearm has the advantage that the cable can be guided along the patient's arm, the wrist does not rub against the cable (8), and in particular the carpal tunnel is also protected.

### TECHNICAL FIELD

The present invention relates to a measuring device for continuous determination of intra-arterial blood pressure on at least one finger of a hand.

### PRIOR ART

The (in particular arterial) blood pressure of a patient is one of the most important measured variables in medical technology, and known, in particular also non-invasive, measuring technology associated with this is extremely diverse. This applies above all to measuring technology for continuously monitoring blood pressure over a prolonged period of time, for example in intensive care medicine, but also in emergency medicine and during surgical interventions.

For reasons of good accessibility, the blood pressure measuring device is often attached to a limb of a patient, for example an applanation tonometric sensor of the radial artery on the forearm or a finger sensor operated in a photoplethysmographic manner according to the so-called “Vascular Unloading Technique” according to Penaz. Such pressure measuring devices are known, for example, from U.S. Pat. Nos. 4,406,289, 4,524,777, 4,726,382, WO 2010/050798 A1, WO 2000/059369 A1, WO 2011/045138 A1, WO 2011/051819 A1, WO 2011/051822 A1, WO 2012/032413 A1, and WO 2017/143366 A1.

In the Vascular Unloading Technique, near-infrared light is radiated into a finger and the pulsatile (pulse-shaped) blood flow (actually the changing blood volume) in the finger is determined from the non-absorbed portion captured by means of a photodetector. For this process, also known as photoplethysmography (PPG), the (near-infrared) light is usually generated using one or more light-emitting diodes (LEDs), which work with one or more wavelengths, and is detected using one or more light-sensitive receiver diodes (photodiodes). Other types of photoreceivers besides diodes are also suitable in principle.

A control system now keeps constant the plethysmographically registered flow (or the detected blood volume) and thus the resulting photoplethysmographic signal (volume signal v(t)) by applying counterpressure in a cuff (cuff pressure) pc(t) on the finger. This counterpressure pc(t) is usually controlled by a high-speed valve or valve system in conjunction with a pump. The relevant control of the valve or valve system is carried out by a control unit, which is preferably implemented using a microcomputer. The main input signals are the PPG signal v(t) and the cuff pressure pc(t). The pressure pc(t) required to keep the PPG signal v(t) constant now corresponds to the intra-arterial blood pressure pa(t).

For this it is necessary that the cuff pressure pc(t) can be changed at least as fast as the intra-arterial blood pressure pa(t) changes, so that the real-time condition is satisfied. The upper limit frequency of pa(t), and thus the highest rate of pressure change, is greater than at least 20 Hz, which is quite a challenge for a pressure control system. From this it follows that the pressure control using a valve or valve system is advantageously disposed in the immediate vicinity of the cuff. If the air lines are too long, there is a risk that this limit frequency condition will be lost due to the low-pass effect of the lines.

A mechanical valve known from U.S. Pat. No. 4,406,289 regulates the counterpressure in the finger cuff with the desired accuracy when it is supplied with a linearly operating pump. The valve is housed in a housing on the distal forearm and thus supplies the finger cuff with the pressure pc(t) via a short hose.

U.S. Pat. No. 4,524,777 describes a pressure generation system for the vascular unloading technique, wherein a constant cuff pressure Pc is also generated with a linear pump and is superimposed with pressure fluctuations Δpc(t) from a “shaker” or a “driving actuator” connected in parallel.

U.S. Pat. No. 4,726,382 discloses a finger cuff for the vascular unloading technique which has hose connections for supplying the cuff pressure pc(t). The length of the air hoses extends to the pressure generation system, which in turn is attached to the distal forearm.

WO 2000/059369 A1 also describes a pressure generation system for the vascular unloading technique. The valve system here comprises a separate inlet valve and a separate outlet valve. While a relatively linear proportional pump must be used in patent specifications U.S. Pat. Nos. 4,406,289 and 4,524,777, this system allows the use of simple, inexpensive pumps, since disruptive harmonics can be eliminated by the arrangement of the valves. Furthermore, the energy consumption of the simple pump can be significantly reduced by the valve principle.

A system for the vascular unloading technique is known from WO 2004/086963 A1 in which the blood pressure can be continuously determined in one finger, while the measurement quality is checked in the adjacent finger (“watch dog” function). After a period of time, the system automatically changes the “measuring finger” to the “monitoring finger”.

WO 2005/037097 A1 describes a control system for the vascular unloading technique having a number of intertwined control loops.

WO 2010/050798 A1 discloses a pressure generation system attached to the distal forearm (“front end”) and having only one valve, to which a finger cuff for the vascular unloading technique can be attached.

In a pressure generation system for the vascular unloading technique described in WO 2011/045138 A1—similar to that known from WO 2000/059369—the energy consumption of the pump is reduced and harmonics can be eliminated.

WO 2011/051819 A1 discloses an implementation of the vascular unloading technique that has been improved by means of digital electronics in order to increase stability and for further miniaturization.

WO 2011/051822 A1 describes a method for the vascular unloading technique in which the measured signals v(t) and pc(t) are processed to increase long-term stability and to determine other hemodynamic parameters. In particular, a method for eliminating effects originating from vasomotor changes in the finger arteries and a method for determining cardiac output (CO) are disclosed.

WO 2012/032413 A1 describes novel finger sensors that have a disposable part for single use. In this case, the cuff that comes into contact with the finger is accommodated in the disposable part for reasons of hygiene, whereas the associated pressure generation and pressure control system is accommodated in a reusable part. Accordingly, a separable pneumatic connection is to be provided here between the disposable part and the reusable part.

As a rule, the pressure generation and pressure control system in the prior art is attached to the distal forearm, proximal to the wrist, which has significant disadvantages: This location is often used for intravenous lines and the intra-arterial access at the distal end of the radial artery should also be free for emergencies. Such accesses can be blocked by the pressure generating and pressure control system and its attachment. In addition, the system can slip or tilt during operation. This can adversely affect how the sensors are seated. The seating of the sensors would also improve if the finger to be measured or the corresponding hand is in a certain rest position.

To overcome this problem, WO 2017/143366 A1 proposes a measuring system for the continuously determining the intra-arterial blood pressure on at least one finger of a hand, having at least one finger sensor, having a plethysmographic system, having at least one light source, preferably LED, having one or several wavelengths and at least one light sensor and at least one inflatable cuff, and having a pressure generation system with at least one valve controlled in real time using the plethysmographic system for generating a pressure in the cuff that is essentially equal to the intra-arterial blood pressure in the finger, wherein the measuring system has a housing with a surface that acts as a resting surface for the at least one finger and the adjacent regions of the palm. The hand rests on a resting surface under which are disposed essential components that were attached to the forearm in conventional systems.

Similar to WO 2012/032413 A1 mentioned above, the cuff is accommodated in a disposable part that can be separated from the housing (and thus from the resting surface). Correspondingly, a separable pneumatic connection between the disposable part and the reusable part is to be provided here, as well.

With the known systems, hygienic problems may occur, as the patient's hand can easily come into contact with reusable parts of the devices. Furthermore, problems may arise during prolonged use, as the patient's hand can become cramped or edges of the device can cut into the hand. It should be noted that patients are often connected to such devices for hours in preparation for surgery, during surgery, especially in a sedated state, and during subsequent monitoring in the recovery room. In the procesThereins, local pressure marks can cause damage to the patient's tissue. Particularly in a sedated state, the patient is unable to control the position of his hand and arm deliberately in order to counteract the formation of pressure marks.

### DESCRIPTION OF THE INVENTION

In view of the limitations existing with conventional systems, it is an object of the present invention to improve measuring devices of the type mentioned at the beginning with regard to hygiene and/or safety and/or comfort for the patient.

According to one aspect of the present invention, this object is achieved with a cuff part according to claim 1. Preferred embodiments of the invention can be implemented according to one of the dependent claims. In particular, the present invention thus provides a measuring device for continuously determining the intra-arterial blood pressure on at least one finger of a hand, which comprises a base part, a cuff part which can be connected to the base part without tools and separated from the base part without tools, comprising at least one ring-like receiving tube for receiving a portion of a finger of a hand passed through the receiving tube and at least one cuff pad arranged in the receiving tube and fillable with a fluid, a radiation source for emitting light into the finger through an optical emission surface, a photodetector for detecting a portion of the light not absorbed in the finger and collected by an optical collector surface, and a cuff pad arranged in the receiving tube and fillable with a fluid, wherein the cuff part further comprises a palm rest for supporting the palm of the hand, and at least one finger rest associated with each of the at least one receiving tube for supporting the portion of the finger projecting beyond the respective receiving tube, and the cuff part projects longitudinally forwardly and rearwardly beyond the base part, the longitudinal direction being defined as the direction of intended insertion of the finger or fingers, respectively, into the receiving tube or the receiving tubes, respectively, wherein the palm rest is arranged behind and the at least one finger rest is arranged in front of the receiving tube. The longitudinal direction thus corresponds to the axial direction of the receiving tube(s).

The palm rest and finger rest(s) ensure that the patient's hand is held as naturally and as fatigue-free as possible during measurement. Since the cuff part projects longitudinally forward and backward beyond the base part, undesirable contact of the patient with the base part is avoided, which is beneficial to the operational safety of the measuring device, hygiene, patient safety and patient comfort.

According to an advantageous embodiment, the base part also does not project beyond the cuff part in the transverse direction over at least the major portion of the length of the base part. As a result, undesirable contact of the patient with the base part can be even better avoided.

According to an advantageous enhancement, the cuff part completely covers the base part in at least one projection plane, in particular a plane parallel to the longitudinal and transverse directions, if a cable connection possibly originating from the base part is not considered to be part of the base part. This results in further increased safety against contact with the base part by the patient.

Preferably, the palm rest comprises a convex curvature in the longitudinal direction on the side intended to face the hand. Such a curvature can significantly improve ergonomics. At least in sections, a convex curvature of the palm rest in the transverse direction can also be advantageously provided (on the side intended to face the hand), which further improves ergonomics. In a rear end region of the palm rest, a concave curvature (on the side intended to face the hand) for the heal of the hand can also advantageously further improve ergonomics.

Preferably, the at least one finger rest comprises a concave curvature in the transverse direction on the side intended to face the hand. This ensures that the respective finger can lie on the finger rest for a long time and largely without movement without cramping.

According to another advantageous enhancement, the entirety of the at least one receiving tube(s), the entirety of the at least one finger rest, and the palm rest are each designed, with respect to a common longitudinal axis, symmetrically on their side intended to face the hand. This allows the measuring device to be used with one and the same cuff part equally for left-handed and right-handed users.

According to a particularly preferred embodiment, the base part comprises a cable connection which is oriented such that the axial direction of a connected cable in its center position is obliquely rearward at an angle between 5 and 45 degrees, preferably between 10 and 30 degrees, relative to the longitudinal direction of the measuring device, when the angle lies in or is projected onto a horizontal angle plane. At the same time or instead, the cable connection may also advantageously be arranged off-center with respect to the width of the base part.

The center position is understood to be the position of the cable that the connection defines when the connected cable extends in a straight line without deflection.

Cable attachment in accordance with the above reduces undesirable contact with the cable. In addition, undesirable loops and kinks can be better avoided when routing the cable: The obliquely or laterally offset attachment of the cable, pointing in the direction of the forearm when the hand is placed as intended, has the advantage that the cable can be routed along the patient's arm, but the wrist area with tendons and vessels is not chafed by the cable, and in particular the carpal tunnel is also protected.

Preferably, the cable connection is arranged in a region of the base part that is covered by the palm rest in a direction perpendicular to the longitudinal and transverse directions.

According to a preferred embodiment, all edges of the receiving tube(s) are rounded and/or chamfered. Thereby, pressure and chafing marks on the fingers as well as the undesirable constriction of blood vessels can be avoided.

Also preferably, all outer edges of the receiving body can be rounded and/or chamfered to prevent pressure and chafing marks on the patient's skin.

In accordance with another aspect of the present invention, there is provided a system comprising a measuring device according to any of the above embodiments and, in addition, at least one further cuff part which is interchangeable, without the need for tools, with the cuff part and which comprises at least one ring-like receiving tube for receiving a portion of a finger of a hand passed through the receiving tube, and at least one cuff pad disposed in the receiving tube and a palm rest for supporting the palm of the hand, and at least one finger rest, which is associated with each of the at least one receiving tube, for supporting the part of the finger projecting beyond the respective receiving tube, wherein the receiving tube and/or the at least one finger rest and/or the palm rest of the cuff part and of the further cuff part are each dimensioned differently from one another, respectively. Thus, ergonomically suitable configurations of the measuring device can be created by changing the cuff parts for patient hands of different sizes.

In principle, every variant of the invention described or indicated in the context of the present application can be particularly advantageous, depending on the economic, technical and possibly medical conditions in the individual case. Unless stated otherwise, or as far as technically feasible in principle, individual features of the embodiments described are interchangeable or can be combined with one another as well as with features known per se from the prior art.

The invention is explained in more detail herein-below in an exemplary manner with reference to the accompanying schematic drawings. The drawings are not to scale; in particular, for reasons of clarity, the proportions of the individual dimensions to one another sometimes do not correspond to the dimensional relationships in actual technical implementations. Corresponding elements are denoted by the same reference signs in the drawings.

### BRIEF DESCRIPTION OF THE DRAWINGS

FIG. 1 shows a measuring device according to the invention in a side view, wherein a cuff part comprising an ergonomic hand rest and a finger-receiving part is placed on a base part containing a pressure control system,

FIG. 2 shows a perspective view of the measuring device of FIG. 1 from obliquely above,

FIG. 3 shows a perspective view of the measuring device of FIG. 1 from obliquely below,

FIG. 4 shows a cuff part like the one shown in FIG. 3 from obliquely below, but without the associated base part, the cuff pads not being shown,

FIG. 5 shows a view of the measuring device from FIG. 1 from behind, i.e. from the left in FIG. 1,

FIG. 6 shows the measuring device of FIG. 1 in plan view.

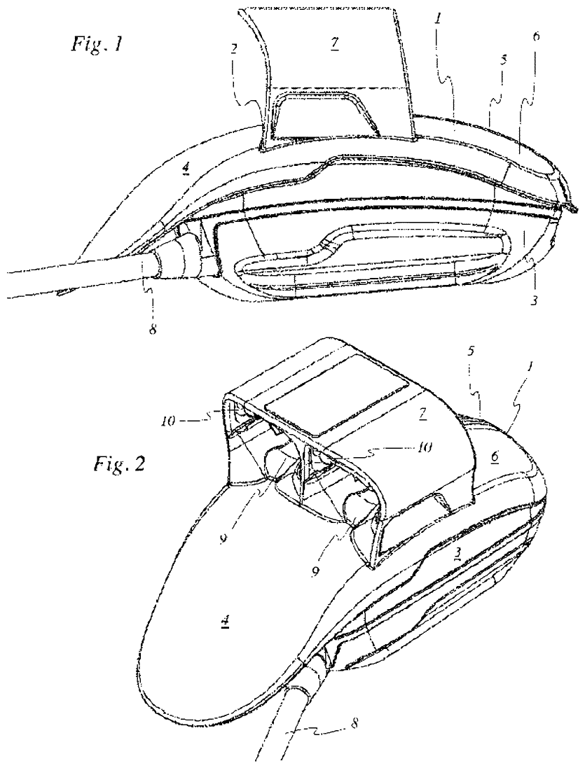

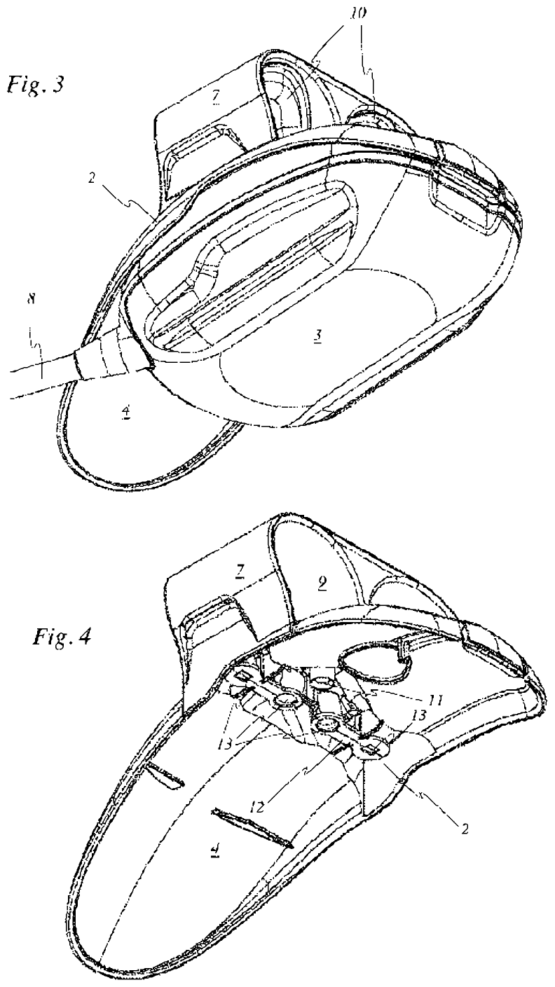

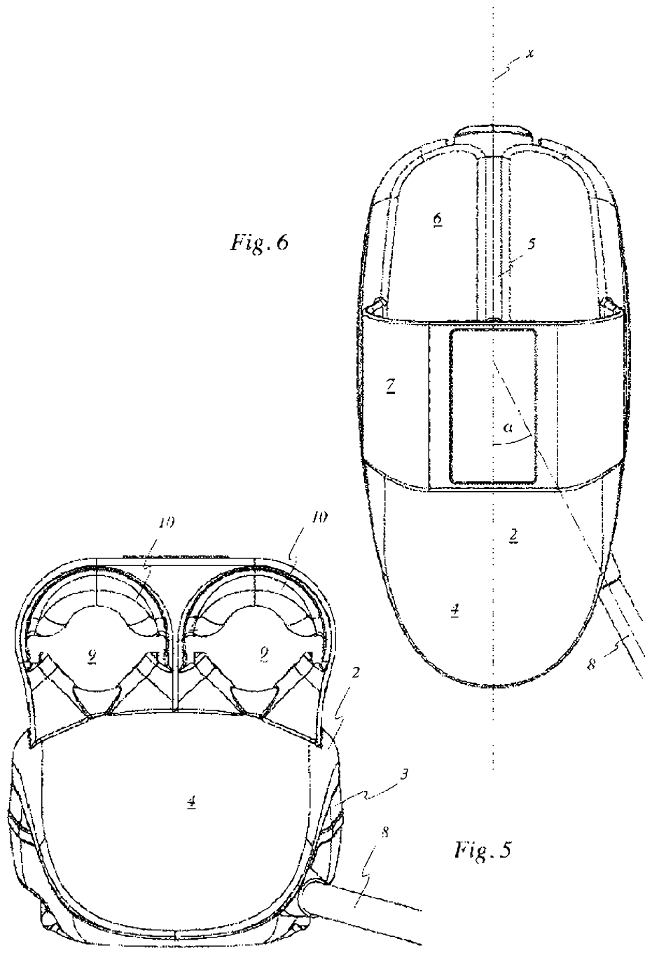

The blood pressure measuring device 1 is designed as a photoplethysmographic measuring system which functions according to the so-called “Vascular Unloading Technique”. Measuring components, i.e. in particular optical and electronic components as well as mechanical components of the pressure generation and pressure control system accommodated in the base part 3, can be implemented in principle in a manner similar to the prior art mentioned at the beginning. The cuff part placed on the base part 3 comprises an ergonomic palm rest 4, two ergonomic finger rests 6 separated from each other by a web 5, and a receiving part 7 comprising two receiving tubes 9 for receiving two fingers, as shown in FIG. 2 and FIG. 5. The finger rests 6 have a concave curvature in their transverse direction so that the fingers can each rest comfortably thereon. The edges at the rims of the receiving tubes 9 are chamfered.

According to the above definition, the left side of FIG. 1 corresponds to the direction indication “rear”, the right side of FIG. 1 corresponds to the direction indication “front”. Accordingly, the upper side of FIG. 6 corresponds to the direction indication “rear”, the lower side of FIG. 6 corresponds to the direction indication “rear”.

As can be seen in FIG. 1, the palm rest 4 comprises a convex curvature in the longitudinal direction at the top (i.e. on the side facing the patient's hand as intended) in order to improve ergonomics. In this region, the palm rest also has a convex curvature in the transverse direction. In an end area (left in FIG. 1), a region curved concavely in the longitudinal direction is provided for the heel of the hand.

The cuff part 2 protrudes beyond the base part 3 both to the front and to the rear. As can be seen from FIG. 6, the cuff part 2 covers the base part 3 almost completely, i.e. to an extent of more than 90%.

As shown in the perspective view of FIG. 2 (viewed from obliquely above at the right in FIG. 1), the receiving part 7 is designed to receive two fingers, which makes alternate measurement on both fingers possible. For reasons of hygiene, the cuff part 2, together with the palm rest 4 and finger rest 6, is designed as a disposable article which is detachably, without tools, attached to the reusable base part 3 by means of a plug-in connection.

The pressure control system in the base part 3 is supplied with compressed air via the obliquely laterally attached cable 8, which points in the direction of the forearm when the hand is placed on the base part as intended. Furthermore, the cable 8 serves to supply energy to the pressure control system (not shown), light sources (not shown) and photodetectors (not shown) or associated control, amplifier and evaluation circuits in the measuring device 1. Measurement data can be output to a patient monitor via a suitable electronic interface through the cable 8.

The lateral attachment of the cable 8, pointing in the direction of the forearm when the hand is placed as intended, has the advantage that the cable can be guided along the patient's arm, but the wrist area with tendons and vessels does not rub against the cable 8, and in particular the carpal tunnel is also protected.

As can be seen in FIG. 6, the central axis of the undeflected cable 8, indicated by a dot and dash line, forms an angle α of here 17 degrees and thus between 10 and 30 degrees with the longitudinal axis x corresponding to the longitudinal direction of the cuff part.

The entirety of the receiving tubes 9, i.e. the receiving part 7, as well as the finger rests 6 and the palm rest 9 are symmetrical with respect to the longitudinal axis x of the cuff part. Thus, the cuff part is equally suitable for the right and left hand.

The two inflatable cuff pads 10 arranged in the receiving tubes 9 are each connected to the pressure generation and pressure control system via a port 11 at the interface between cuff part 2 and base part 3. In the illustration of cuff part 2 in FIG. 4, one of the two ports 11 is hidden by the crosspiece 12, which carries light guides 13 through which light is conducted from the light sources arranged in base part 3 to the fingers or from the fingers to the photosensors arranged in base part 3. A valve device is preferably located at port 11, so that port 11 is flush with the housing of base part 3 on the base part side when base part 3 and cuff part 2 are not connected to each other.

1. A measuring device for continuous determination of intra-arterial blood pressure on at least one finger of a hand, which comprises:
a base part,
a cuff part which is connectable to the base part without tools and separatable from the base part without tools and which comprises at least one ring-like receiving tube for receiving a portion of a finger of a hand passed through the receiving tube, and at least one cuff pad which is arranged in the receiving tube and fillable with a fluid,
a radiation source for emitting light into the finger through an optical emission surface,
a photodetector for detecting a portion of the light collected by an optical collector surface and not absorbed in the finger, and
a pressure control system disposed at least partially in the base portion for controlling a fluid pressure in the cuff pad in response to the detected non-absorbed portion of the light,
wherein the cuff part further comprises a palm rest for supporting the palm of the hand and at least one finger rest associated with each of the at least one receiving tube, respectively, for supporting the portion of the finger protruding beyond the respective receiving tube,
and the cuff part projects beyond the base part in the longitudinal direction towards the front and towards the rear, the longitudinal direction being defined as the direction of intended insertion of the finger into the receiving tube or of the fingers into the receiving tubes, the palm rest being arranged behind and the at least one finger rest being arranged in front of the receiving tube.

2. The measuring device according to claim 1, wherein the base part does not project beyond the cuff part in the transverse direction over at least the major part of the length of the base part.

3. The measuring device according to claim 2, wherein the cuff part completely covers the base part in at least one projection plane, in particular a projection plane parallel to both the longitudinal and transverse directions, if a cable connection possibly extending from the base part is not considered part of the base part.

4. The measuring device according to any one of the preceding claims, wherein the palm rest comprises a convex curvature in longitudinal direction on the side intended to face the hand.

5. The measuring device according to any one of the preceding claims, wherein the at least one finger rest comprises a respective concave curvature in the transverse direction on the side intended to face the hand.

6. The measuring device according to any one of the preceding claims, wherein
a) the entirety of the at least one receiving tube(s),
b) the entirety of the at least one finger rest, and
c) the palm rest
are, on their side intended to face the hand, each symmetrical with respect to a common longitudinal axis.

7. The measuring device according to any one of the preceding claims, wherein
the base part comprises a cable connection
which is oriented in such a way that the axial direction of a connected cable in its central position, i.e. without deflection of the cable, points obliquely backwards relative to the longitudinal direction of the measuring device at an angle of between 5 and 45 degrees when the angle lies in a horizontal angle plane or is projected onto a horizontal angle plane,
and/or which is off-center with respect to the width of the base part.

8. The measuring device according to claim 7, wherein the angle is between 10 and 30 degrees.

9. The measuring device according to claim 7 or claim 8, wherein the cable connection is arranged in the region of the base part which is covered by the palm rest in a direction perpendicular to the longitudinal and transverse directions.

10. The measuring device according to one of the preceding claims, wherein all edges of the receiving tube(s) are rounded and/or chamfered.

11. The measuring device according to any one of the preceding claims, wherein all outer edges of the receiving tube(s) are rounded and/or chamfered.

12. A system comprising
a measuring device according to any one of claims 1-11, and
at least one further cuff part interchangeable with the cuff part without tools, comprising at least one ring-like receiving tube for receiving a portion of a finger of a hand passed through the receiving tube, and at least one fluid-fillable cuff pad arranged in the receiving tube, a palm rest for supporting the palm of the hand, and at least one finger rest associated with each of the at least one receiving tube for supporting the portion of the finger extending beyond the respective receiving tube,
wherein the receiving tube and/or the at least one finger rest and/or the palm rest of the cuff part and of the further cuff part are dimensioned differently from one another, respectively.
